(19) United States
(12) Patent Application Publication
(10) Pub. No.: US 2025/0288336 A1
(43) Pub. Date: Sep. 18, 2025

(54) SACROILIAC JOINT STABILIZATION SYSTEMS AND METHODS

(71) Applicant: FloSpine, LLC, Boca Raton, FL (US)

(72) Inventors: Peter M. HARRIS, Boca Raton, FL (US); Lawrence CHARNOW, Wellington, FL (US)

(73) Assignee: FloSpine, LLC, Boca Raton, FL (US)

(21) Appl. No.: 19/082,908

(22) Filed: Mar. 18, 2025

Related U.S. Application Data
(60) Provisional application No. 63/566,550, filed on Mar. 18, 2024.

(51) Int. Cl. A61B 17/86; A61F 2/30

(52) U.S. Cl. CPC A61B 17/8685; A61F 2/30988; A61F 2002/30995

(57) ABSTRACT

A sacroiliac joint stabilization system may be configured to stabilize a sacroiliac joint. The sacroiliac joint stabilization system may include a fastener having a threaded portion, and an implant having a body including a body height and an aperture extending along the body height, a first wing, a second wing, a third wing, and a fourth wing. The first wing, the second wing, the third wing, and the fourth wing may be arranged relative to each other to define an X shape, the threaded portion may be configured to advance through the aperture and engage a sacrum and an ilium, the first wing and the second wing may be configured to be embedded in the sacrum, and the third wing and the fourth wing may be configured to be embedded in the ilium.

### CROSS REFERENCE TO RELATED APPLICATIONS

The present application claims the benefit of U.S. Provisional Application No. 63/566,550 filed on Mar. 18, 2024, entitled “SACROILIAC JOINT STABILIZATION SYSTEMS AND METHODS”, which is incorporated by reference as though set forth herein in its entirety.

### TECHNICAL FIELD

The present disclosure relates generally to surgical systems and methods, and more particularly to systems and methods for stabilization of the sacroiliac joint.

### BACKGROUND

The sacroiliac (SI) joint plays a crucial role in transferring load between the spine and the pelvis while allowing limited movement to absorb stress. Dysfunction of the SI joint is a recognized source of lower back pain, often resulting from trauma, degeneration, or inflammatory conditions. Traditional treatments for SI joint dysfunction include physical therapy, pain management, and, in severe cases, surgical intervention.

Existing implants for SI joint stabilization are typically inserted through lateral or transiliac approaches. While effective, these techniques have limitations, including challenges in achieving optimal implant placement, risk to neurovascular structures, and potential for inadequate stabilization in certain patient anatomies.

A posterior approach to SI joint stabilization offers direct access to the joint with potential advantages such as reduced soft tissue disruption, improved visualization, and the ability to place implants in a mechanically advantageous orientation.

Accordingly, there is a need for an improved sacroiliac implant specifically configured for a posterior approach, enabling enhanced stabilization of the sacrum relative to the ilium while minimizing surgical risks and procedural complexity.

### SUMMARY

The various systems and methods of the present disclosure have been developed in response to the present state of the art, and in particular, in response to the problems and needs in the art that have not yet been fully solved by currently available sacroiliac joint stabilization systems and methods.

In some embodiments, a sacroiliac joint stabilization system may be configured to stabilize a sacroiliac joint. The sacroiliac joint stabilization system may include a fastener having a threaded portion, and an implant having a body including a body height and an aperture extending along the body height, a first wing, a second wing, a third wing, and a fourth wing. The first wing, the second wing, the third wing, and the fourth wing may be arranged relative to each other to define an X shape, the threaded portion may be configured to advance through the aperture and engage a sacrum and an ilium, the first wing and the second wing may be configured to be embedded in the sacrum, and the third wing and the fourth wing may be configured to be embedded in the ilium.

In the sacroiliac joint stabilization system of any preceding paragraph, the first wing, the second wing, the third wing and the fourth wing may extend radially from the body.

In the sacroiliac joint stabilization system of any preceding paragraph, the first wing may include a first angled portion forming a first acute angle relative to the first wing so that the first angled portion may be angled toward the second wing, the second wing may include a second angled portion forming a second acute angle relative to the second wing so that the second angled portion may be angled toward the first wing, the third wing may include a third angled portion forming a third acute angle relative to the third wing so that the third angled portion may be angled toward the fourth wing, and the fourth wing may include a fourth angled portion forming a fourth acute angle relative to the fourth wing so that the fourth angled portion may be angled toward the third wing.

In the sacroiliac joint stabilization system of any preceding paragraph, the first angled portion may include a first sharp tapered edge, the second angled portion may include a second sharp tapered edge, the third angled portion may include a third sharp tapered edge, the fourth angled portion may include a fourth sharp tapered edge, and the first sharp tapered edge, the second sharp tapered edge, the third sharp tapered edge, and the fourth sharp tapered edge may be configured so that, with the implant embedded in the sacrum and the ilium, the first angled portion, the second angled portion, the third angled portion, and the fourth angled portion may urge the sacrum towards the ilium and the ilium towards the sacrum.

In the sacroiliac joint stabilization system of any preceding paragraph, the first wing may include a first sharp edge, the second wing may include a second sharp edge, the third wing may include a third sharp edge, the fourth wing may include a fourth sharp edge, the first sharp edge and the second sharp edge may be configured to penetrate the sacrum, and the third sharp edge and the fourth sharp edge may be configured to penetrate the ilium.

In the sacroiliac joint stabilization system of any preceding paragraph, the fastener may further include a head portion comprising a drive feature, and a shaft portion between the threaded portion and the head portion, wherein the aperture may be configured to receive the shaft portion, and, with the shaft portion received in the aperture, the fastener may be rotatable relative to the implant.

In the sacroiliac joint stabilization system of any preceding paragraph, the first wing may include a first height that is generally equal to the body height, the second wing may include a second height that is generally equal to the body height, the third wing may include a third height that is generally equal to the body height, and the fourth wing may include a fourth height that is generally equal to the body height.

In some embodiments, a sacroiliac joint stabilization system may be configured to stabilize a sacroiliac joint between a sacrum and an ilium. The sacroiliac joint stabilization system may include a fastener including a threaded portion, a head portion including a drive feature, and a shaft portion between the threaded portion and the head portion, and an implant including a body including a body height, an aperture extending along the body height and including an internal thread, a first wing including a first height that may be generally equal to the body height, a second wing including a second height that may be generally equal to the body height, a third wing including a third height that may be generally equal to the body height, and a fourth wing including a fourth height that may be generally equal to the body height. The first wing, the second wing, the third wing and the fourth wing may be arranged relative to each other to define an X shape, the threaded portion may be configured to engage the internal thread and may threadably advance through the aperture, the aperture may be configured to receive the shaft portion, and, with the shaft portion received in the aperture, the fastener may be rotatable relative to the implant.

In the sacroiliac joint stabilization system of any preceding paragraph, the threaded portion may be configured to advance through the aperture and engage the sacrum and the ilium, the first wing and the second wing may be configured to be embedded in the sacrum, and the third wing and the fourth wing may be configured to be embedded in the ilium.

In the sacroiliac joint stabilization system of any preceding paragraph, the first wing, the second wing, the third wing and the fourth wing may extend radially from the body.

In the sacroiliac joint stabilization system of any preceding paragraph, the first wing may include a first angled portion forming a first acute angle relative to the first wing so that the first angled portion may be angled toward the second wing, the second wing may include a second angled portion forming a second acute angle relative to the second wing so that the second angled portion may be angled toward the first wing, the third wing may include a third angled portion forming a third acute angle relative to the third wing so that the third angled portion may be angled toward the fourth wing, and the fourth wing may include a fourth angled portion forming a fourth acute angle relative to the fourth wing so that the fourth angled portion may be angled toward the third wing.

In the sacroiliac joint stabilization system of any preceding paragraph, the first angled portion may include a first sharp tapered edge, the second angled portion may include a second sharp tapered edge, the third angled portion may include a third sharp tapered edge, the fourth angled portion may include a fourth sharp tapered edge, and the first sharp tapered edge, the second sharp tapered edge, the third sharp tapered edge, and the fourth sharp tapered edge may be configured so that, with the implant embedded in the sacrum and the ilium, the first angled portion, the second angled portion, the third angled portion, and the fourth angled portion may urge the sacrum towards the ilium and the ilium towards the sacrum.

In the sacroiliac joint stabilization system of any preceding paragraph, the first wing may include a first sharp edge, the second wing may include a second sharp edge, the third wing may include a third sharp edge, the fourth wing may include a fourth sharp edge, the first sharp edge and the second sharp edge may be configured to penetrate the sacrum, and the third sharp edge and the fourth sharp edge may be configured to penetrate the ilium.

In the sacroiliac joint stabilization system of any preceding paragraph, the threaded portion may be configured to advance through the aperture and engage the sacrum and the ilium.

In some embodiments, a sacroiliac joint stabilization system may be configured to stabilize a sacroiliac joint between a sacrum and an ilium. The sacroiliac joint stabilization system may include an implant including a first wing having a first sharp edge, a second wing having a second sharp edge, a third wing having a third sharp edge, and a fourth wing having a fourth sharp edge. The first wing, the second wing, the third wing and the fourth wing may be arranged relative to each other to define an X shape, the first sharp edge and the second sharp edge may be configured to penetrate the sacrum, and the third sharp edge and the fourth sharp edge may be configured to penetrate the ilium.

In the sacroiliac joint stabilization system of any preceding paragraph, the first wing and the second wing may be configured to be embedded in the sacrum, and the third wing and the fourth wing may be configured to be embedded in the ilium.

In the sacroiliac joint stabilization system of any preceding paragraph, with the first wing and the second wing embedded in the sacrum, and the third wing and the fourth wing embedded in the ilium, the implant may be configured to urge the sacrum towards the ilium, and the ilium towards the sacrum.

In the sacroiliac joint stabilization system of any preceding paragraph, the implant may further include a body having an aperture extending along a body height of the body. The first wing, the second wing, the third wing and the fourth wing may extend radially from the body, the first wing may include a first height that is generally equal to the body height, the second wing may include a second height that is generally equal to the body height, the third wing may include a third height that is generally equal to the body height, and the fourth wing may include a fourth height that is generally equal to the body height.

In the sacroiliac joint stabilization system of any preceding paragraph, the first wing may include a first angled portion forming a first acute angle relative to the first wing so that the first angled portion may be angled toward the second wing, the second wing may include a second angled portion forming a second acute angle relative to the second wing so that the second angled portion may be angled toward the first wing, the third wing may include a third angled portion forming a third acute angle relative to the third wing so that the third angled portion may be angled toward the fourth wing, and the fourth wing may include a fourth angled portion forming a fourth acute angle relative to the fourth wing so that the fourth angled portion may be angled toward the third wing.

In the sacroiliac joint stabilization system of any preceding paragraph, the sacroiliac joint stabilization system may further include a fastener having a threaded portion, a head portion including a drive feature, and a shaft portion between the threaded portion and the head portion. The implant may further include a body having an aperture extending along a body height of the body, the aperture may be configured to receive the shaft portion, and, with the shaft portion received in the aperture, the fastener may be rotatable relative to the implant.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings.

FIG. 1 is a perspective view of a sacroiliac joint stabilization system according to an embodiment of the present disclosure.

FIG. 2 is an exploded perspective view of the sacroiliac joint stabilization system of FIG. 1.

FIG. 3A is a top view of the sacroiliac joint stabilization system of FIG. 1.

FIG. 3B is a front view of the sacroiliac joint stabilization system of FIG. 1.

FIG. 3C is a perspective view of the sacroiliac joint stabilization system of FIG. 1.

FIG. 4A is a top view of a fastener of the sacroiliac joint stabilization system of FIG. 1.

FIG. 4B is a front view of the fastener of FIG. 4A.

FIG. 4C is a perspective view of the fastener FIG. 4A.

FIG. 5A is a top view of an Implant of the sacroiliac joint stabilization system of FIG. 1.

FIG. 5B is a front view of the Implant of FIG. 5A.

FIG. 5C is a perspective view of the Implant of FIG. 5A.

FIG. 6A is a perspective view of a driver assembly according to an embodiment of the present disclosure.

FIG. 6B is a perspective view of a handle of the driver assembly of FIG. 6A.

FIG. 6C is a perspective view of a shaft of the driver assembly of FIG. 6A.

FIG. 7 is a perspective view of a straight chisel body according to an embodiment of the present disclosure.

FIG. 8 is a perspective view of a straight chisel assembly and a forked dilator tube according to an embodiment of the present disclosure.

FIG. 9A is a perspective view of the forked dilator tube of FIG. 8.

FIG. 9B is a front view of the forked dilator tube of FIG. 8.

FIG. 10 is a perspective view of a step drill according to an embodiment of the present disclosure.

FIG. 11 is a perspective view of an Implant inserter according to an embodiment of the present disclosure.

FIG. 12 is a front view of the sacroiliac joint stabilization system of FIG. 1 implanted in a sacroiliac joint.

FIG. 13A is a perspective view of a sacroiliac joint stabilization system according to an embodiment of the present disclosure.

FIG. 13B is a perspective view of an Implant of the sacroiliac joint stabilization system of FIG. 13A.

FIG. 13C is an opposite perspective view of the Implant of FIG. 13B.

FIG. 13D is a side view of the Implant of FIG. 13B.

FIG. 13E is a top view of the Implant of FIG. 13B.

FIG. 14 is a front view of the sacroiliac joint stabilization system of FIG. 13A implanted in a sacroiliac joint.

FIG. 15A through FIG. 15F are a method for implanting a sacroiliac joint stabilization system into a sacroiliac joint according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

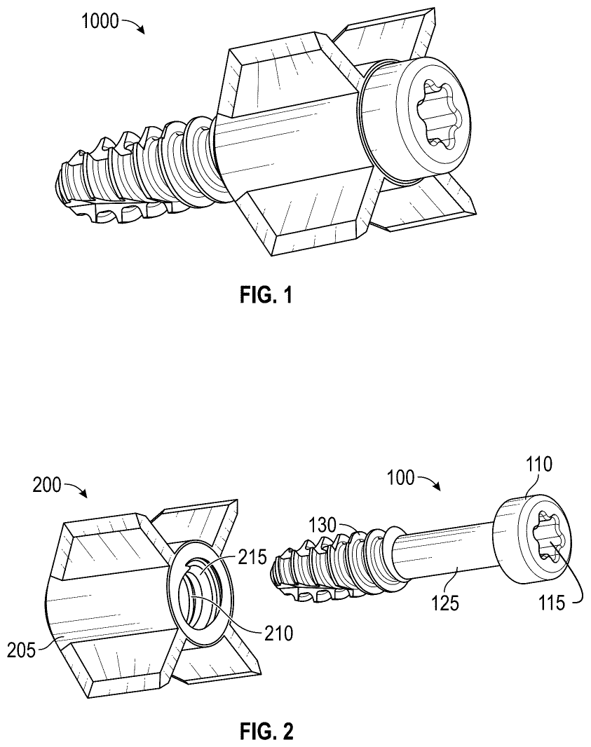

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIG. 1 through FIG. 15F, is not intended to limit the scope of the claims, but is merely representative exemplary of exemplary embodiments of the disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

FIG. 1 is a perspective view of a sacroiliac joint stabilization system 1000 according to an embodiment of the present disclosure. The sacroiliac joint stabilization system 1000 may be implanted within a sacroiliac joint and may be configured to stabilize the sacroiliac joint. The sacroiliac joint stabilization system 1000 may be configured to minimize movement of an ilium 820 with respect to an adjacent sacrum 810. The sacroiliac joint stabilization system 1000 may include a first portion configured for implantation in the ilium 820 and a second portion configured for implantation in the sacrum 810. Additionally, the sacroiliac joint stabilization system may also include a central portion configured for implantation within the sacroiliac joint 800. The central portion may be proximate both the ilium 820 and the sacrum 810. The sacroiliac joint stabilization system 1000 may be configured for a posterior surgical approach.

FIG. 2 is an exploded perspective view of the sacroiliac joint stabilization system 1000. The sacroiliac joint stabilization system may include a fastener 100 and an Implant 200. The fastener 100 may include a threaded portion 130, a head portion 110 including a drive feature 115, and a shaft portion 125 between the threaded portion 130 and the head portion 110. The Implant 200 may include a body 205, an aperture 210 extending along a longitudinal axis of the body 205, and an internal thread 215. The fastener 100 may be received in the Implant 200 so that the body 205 may be proximate to the head portion 110.

The internal thread 215 may be configured to allow the threaded portion 130 to threadably pass through the body 205. The shaft portion 125 may then be received in the aperture 210 so that the shaft portion 125 may freely rotate relative to the Implant 200.

Alternatively, the aperture 210 may be larger than the threaded portion 130 and the internal thread 215 may be omitted so that the threaded portion 130 may pass though the body 205. The shaft portion 125 may then rotate freely relative to the Implant 200.

The threaded portion 130 may be configured to engage a first bone of a joint and a second bone of the joint. The threaded portion 130 may be configured to advance through the aperture 210 and engage a sacrum and an ilium.

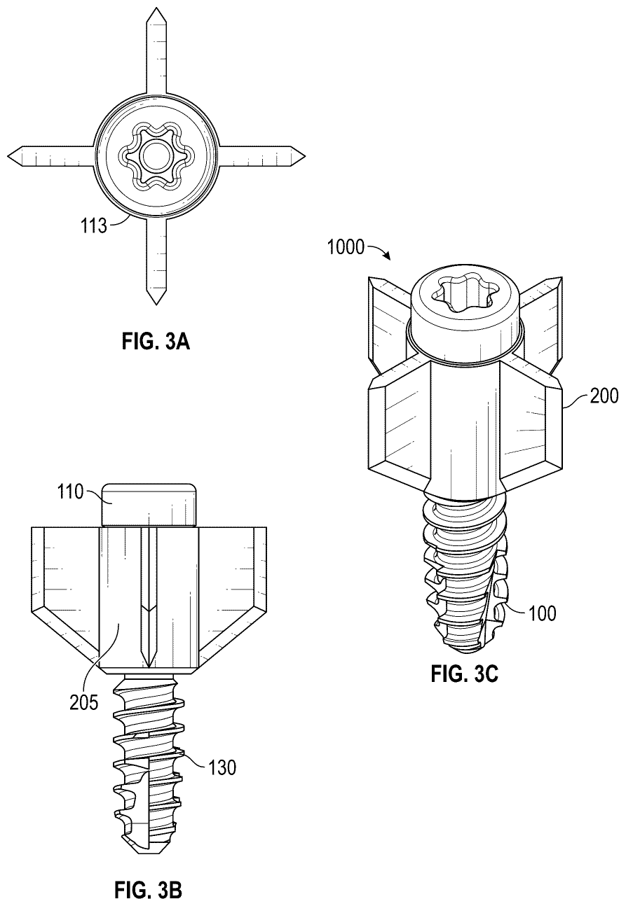

FIG. 3A is a top view of the sacroiliac joint stabilization system 1000, FIG. 3B is a front view of the sacroiliac joint stabilization system 1000, and FIG. 3C is a perspective view of the sacroiliac joint stabilization system 1000. The head portion 110 may be larger than the aperture 210 thereby preventing the Implant 200 from disengaging from the proximal end of the fastener 100.

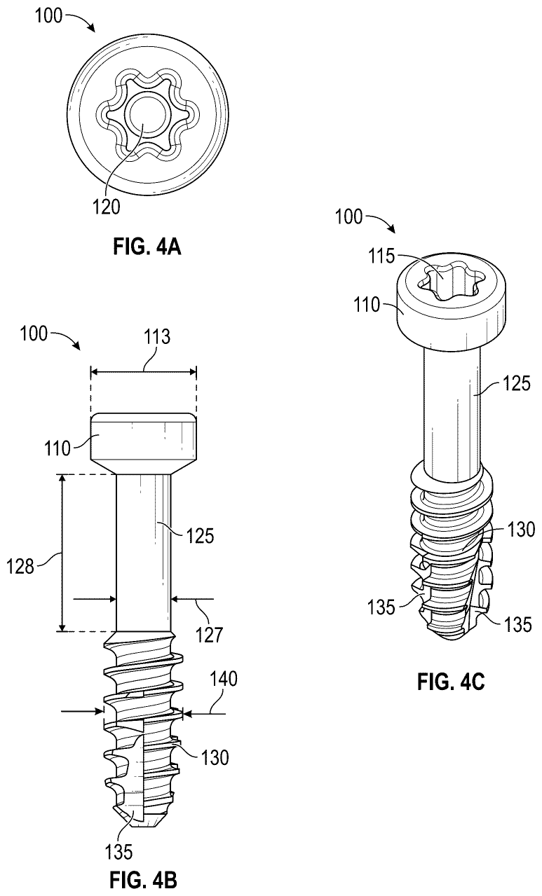

FIG. 4A is a top view of the fastener 100 of the sacroiliac joint stabilization system 1000, FIG. 4B is a front view of the fastener 100 of the sacroiliac joint stabilization system 1000, and FIG. 4C is a perspective view of the fastener 100 of the sacroiliac joint stabilization system 1000. The fastener 100 may be configured to threadably engage a bone and/or a joint and to secure the Implant 200 in one or more bones and/or a joint.

The fastener 100 may include a head portion 110 with a head diameter 113, a drive feature 115, and a cannulation 120. The head portion 110 may be at the proximal end of the fastener 100 and may include the drive feature 115. The head diameter 113 may be larger than the aperture 210 of the Implant 200 so that the Implant 200 is retained on the shaft portion 125 after the fastener 100 is received in the Implant 200.

The drive feature 115 may be shaped to receive a corresponding tip portion 350 of a driver assembly 300. As shown, the drive feature 115 may have a hexalobe geometry or the like; this geometry may match that of the tip portion 350 so that the driver assembly 300 may impart torque to the fastener 100 through rotation of the driver assembly 300. In alternative embodiments, the drive feature 115 may have a different geometry or may be replaced by one or more positive features such as a boss or any other driver tip geometry known in the art.

The cannulation 120 may be an internal diameter extending along the entire length of the fastener 100 and generally along the same longitudinal axis as the head portion 110, the shaft portion 125 and/or, the threaded portion 130. The cannulation 120 may be sized to slidably receive a k-wire 850 (or guide wire).

The fastener 100 may also include a shaft diameter 127, a shaft length 128, one or more cutting flutes 135, and a thread diameter 140. The threaded portion 130 may include a thread profile configured to engage a portion of bone and/or a joint. The distal end of the fastener 100 may include one or more cutting flutes 135. The one or more cutting flutes 135 may be configured to interrupt the thread diameter 140 along the threaded portion 130 to decrease the torque required to drive the fastener 100 into a bone and/or a joint.

The one or more cutting flutes 135 may also be deep enough to interrupt the minor diameter of the threaded portion 130. The one or more cutting flutes 135 may extend along the length of the threaded portion 130 in a helical manner. Alternatively, the one or more cutting flutes 135 may extend along the length of the threaded portion 130 in a straight manner.

The threaded portion 130 may have a thread diameter 140 that may be larger than the shaft diameter 127. Additionally, the thread diameter 140 may be larger than the aperture 210 of the Implant 200. The shaft diameter 127 may be smaller than the aperture 210 of the Implant 200.

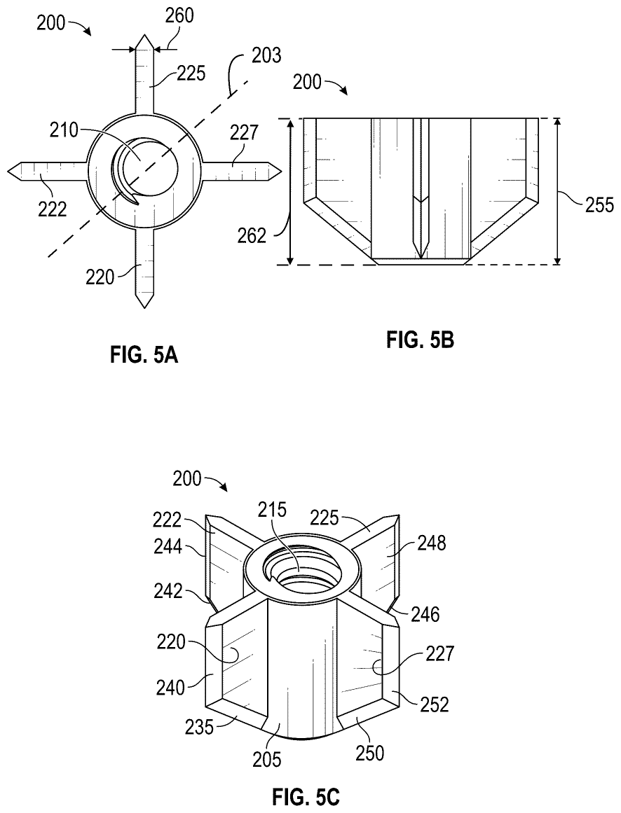

FIG. 5A is a top view of the Implant 200 of the sacroiliac joint stabilization system 1000, FIG. 5B is a front view of the Implant 200 of the sacroiliac joint stabilization system 1000, and FIG. 5C is a perspective view of the Implant 200 of the sacroiliac joint stabilization system 1000. The Implant 200 may be configured to span a sacroiliac joint 800 and engage a portion of a sacrum 810 and a portion of an ilium 820. The Implant 200 may further be configured to minimize movement of an ilium 820 with respect to an adjacent sacrum 810. Additionally, the Implant 200 may be configured to receive a fastener 100 through the aperture 210 in the body 205. The Implant 200 may further be configured to receive the fastener 100 through rotation of the fastener 100 but without rotation of the Implant 200.

The Implant 200 may include a body 205, a body height 255, and an aperture 210 extending along the body height 255 and including an internal thread 215. The internal thread 215 may include a thread pitch, thread form, and thread diameter compatible with the threaded portion 130 and thread diameter 140 of the fastener 100. The internal thread 215 may be configured to allow the threaded portion 130 of the fastener 100 to threadably pass though the aperture 210. The body height 255 may be less than or equal to the shaft length 128 of the fastener 100 so that, after the threaded portion 130 of the fastener 100 passes through the aperture 210, the shaft portion 125 of the fastener 100 may freely rotate within the Implant 200 without the threaded portion 130 engaging the internal thread 215. The body height 255 may be between 10 mm and 15 mm. More specifically, the body height 255 may be between 12 mm and 13 mm. Even more specifically, the body height 255 may be approximately 12.5 mm.

The Implant 200 may also include a first wing 220 having a first sharp edge 235 and a first sharp side edge 240, a second wing 222 having a second sharp edge 242 and a second sharp side edge 244, a third wing 225 having a third sharp edge 246 and a third sharp side edge 248, and a fourth wing 227 having a fourth sharp edge 250 and a fourth sharp side edge 252. The first sharp edge 235, the second sharp edge 242, the third sharp edge 246, and the fourth sharp edge 250 may be configured to penetrate a bone.

More specifically, the first sharp edge and the second sharp edge may be configured to penetrate the sacrum and the third sharp edge and the fourth sharp edge may be configured to penetrate the ilium. Additionally, or alternatively, the first wing 220 and the second wing 222 may be configured to be embedded in the sacrum 810. The third wing 225 and the fourth wing 227 may be configured to be embedded in the ilium 820.

Each of the first wing 220, the second wing 222, the third wing 225, and the fourth wing 227 may further have a wing width 260. The wing width 260 may be generally equal for each of the first wing 220, the second wing 222, the third wing 225, and the fourth wing 227. Alternatively, the wing width 260 may differ for each of the first wing 220, the second wing 222, the third wing 225, and the fourth wing 227. The wing width 260 may be between 1.0 mm and 1.5 mm. More specifically, the wing width 260 may be between 1.2 mm and 1.3 mm.

The first wing 220, the second wing 222, the third wing, and the fourth wing 227 may include a wing height 262 that may be generally equal to the body height 255.

Each of the first wing 220, the second wing 222, the third wing 225, and the fourth wing 227 may extend radially from the body 205. The first wing 220, the second wing 222, the third wing 225, and the fourth wing 227 may be generally equally spaced around a perimeter of the body whereby the angle between any two adjacent wings is approximately 90°. Additionally, or alternatively, the first wing, the second wing, the third wing, and the fourth wing may be arranged relative to each other to define an X shape.

Alternatively, the angle between the first wing 220 and the second wing 222 and the angle between the third wing 225 and the fourth wing 227 may each be less than 90° and the angle between the second wing 222 and the third wing 225 and the angle between the fourth wing 227 and the first wing 220 may each be greater than 90° whereby the sum of the four angles equals 360°. Additionally, or alternatively, the angle between the first wing 220 and the second wing 222 may be different than the angle between the third wing 225 and the fourth wing 227.

Additionally, or alternatively, one or more of the first wing 220, the second wing 222, the third wing 225, and the fourth wing 227 may be colinear with a secant of the body 205 and not colinear with a diameter of the body 205.

Additionally, or alternatively, the body 205 may be configured as a polygon or other non-circular shape whereby each of the first wing 220, the second wing 222, the third wing 225, and the fourth wing 227 may extend from a face or a vertex of the polygon. In an embodiment, a first wing 220 and a second wing 222 may be positioned on a first side of a center plane 203 that bisects the center of a body 205, along a longitudinal axis of the body 205, and a third wing 225 and a fourth wing 227 may be positioned on a second side of the center plane 203.

The Implant 200 may be configured to be embedded into the sacrum 810 and ilium 820 by orienting the Implant 200 such that the first wing 220 and the second wing 222 face the sacrum 810 and the third wing 225 and fourth wing 227 face the ilium 820 and then pressing (or impacting) the Implant 200 such that the first sharp edge 235 and the second sharp edge 242 penetrate the sacrum 810 and the third sharp edge 246 and fourth sharp edge 250 penetrate the ilium 820. The Implant 200 may be further pressed (or impacted) until the Implant 200 is fully embedded within the sacrum 810 and/or the ilium 820.

Alternatively, the Implant 200 may not be fully embedded within the sacrum 810 and/or the ilium 820, the proximal end of the Implant 200 may protrude above the surface of the sacrum 810 and/or the ilium 820. Additionally, or alternatively, the sacroiliac joint stabilization system 1000 may be configured so that, as the fastener 100 threadably engages a portion of bone, the head portion 110 exerts a linear force on the body 205 that may result in embedding the Implant 200 deeper into the sacrum 810 and/or the ilium 820.

The Implant 200 may be positioned such that the acute angled formed between the first wing 220 and the sacroiliac joint 800 may be generally equal to the acute angle formed between the second wing 222 and the sacroiliac joint 800. Alternatively, the Implant 200 may be positioned such that the acute angled formed between the first wing 220 and the sacroiliac joint 800 is generally less than the acute angle formed between the second wing 222 and the sacroiliac joint 800.

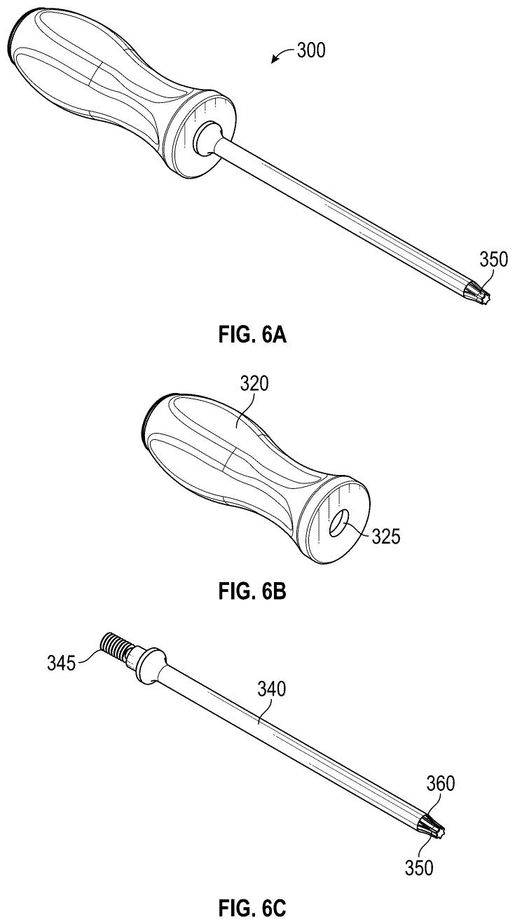

FIG. 6A is a perspective view of a driver assembly 300 according to an embodiment of the present disclosure. FIG. 6B is a perspective view of a handle 320 of the driver assembly 300. FIG. 6C is a perspective view of a shaft 340 of the driver assembly 300. The driver assembly 300 may be configured to engage the fastener 100 to provide torque to the fastener 100 resulting in threaded engagement of the fastener 100 and a bone portion. The driver assembly 300 may include a handle 320 having a connection portion 325. The driver assembly 300 may further include a shaft 340 having a connection portion 345 and a tip portion 350 including a retaining feature 360.

The connection portion 325 of the handle 320 may be configured to receive the connection portion 345 of the shaft 340. The connection portion 325 may be an internal thread with a pitch and a pitch diameter that corresponds to the connection portion 345, which may be an external thread. Alternatively, the connection portion 325 and the connection portion 345 may have complimentary features that establish a secure connection between the handle 320 and the shaft 340 such that the handle 320 may impart sufficient torque to the shaft 340 to drive the fastener 100 into a portion of bone.

As shown, tip portion 350 may have a hexalobe geometry or the like; this geometry may match that of the drive feature 115 of the fastener 100 so that the driver assembly 300 may impart torque to the fastener 100 through rotation of the driver assembly 300. In alternative embodiments, the tip portion 350 may have a different geometry or may be replaced by one or more features such as a socket or any other driver tip geometry known in the art.

The tip portion 350 may further include a retaining feature 360 whereby, when the tip portion 350 is engaged with the drive feature 115 of the fastener 100, the fastener 100 is sufficiently retained on the tip portion 350 so that the fastener 100 may be introduced into a surgical site with out unintentionally disengaging from the tip portion 350. The retaining feature 360 may be included in one or more lobes of the hexalobe geometry of the tip portion 350. The retaining feature 360 may be configured as an increase in the concave portion of the hexalobe geometry. The increase may be in the form of an angle, a radius, or a combination of the two.

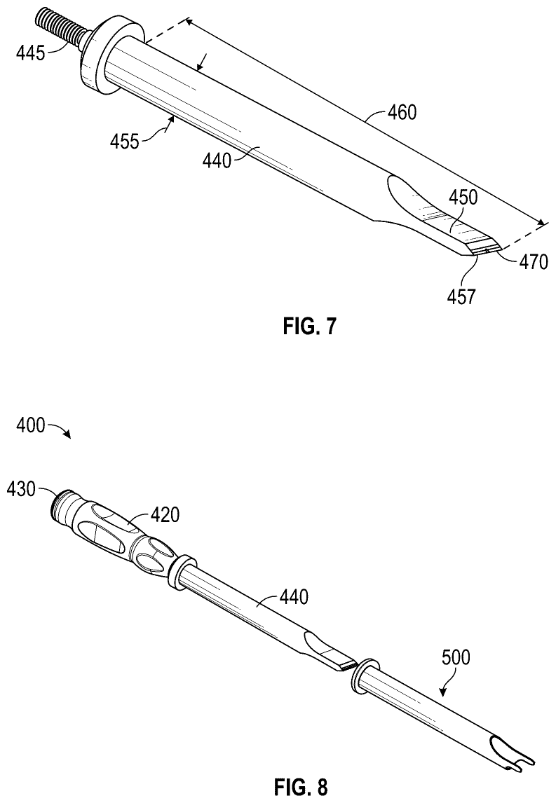

FIG. 7 is a perspective view of a body 440 of a straight chisel assembly 400 according to an embodiment of the present disclosure. FIG. 8 is a perspective view of the straight chisel assembly 400 and a forked dilator tube 500 according to an embodiment of the present disclosure. The straight chisel assembly 400 may be configured to distract the sacroiliac joint 800. The straight chisel assembly may further be configured to facilitate introduction of the distal end of the forked dilator tube into the sacroiliac joint 800. The straight chisel assembly 400 may introduced into a surgical site by advancing over a k-wire 850.

The straight chisel assembly 400 may include a handle 420 having an impaction end 430 and a body 440 having a connection portion 445. The handle 420 may be configured to receive the connection portion 445 of the shaft 340. The connection portion 445 may be an external thread with a pitch and a pitch diameter that corresponds to a connection portion within the handle 420, which may be an internal thread. Alternatively, the connection portion 445 and the handle 420 may have complimentary features that establish a secure connection between the handle 420 and the body 440.

The body 440 may also include a tip portion 450 having a leading angle 457, a shaft diameter 455, a shaft length 460, and a cannulation 470. The body 440 may be configured to be received within the forked dilator tube 500. The leading angle 457 may be configured to facilitate introduction of the tip portion 450 into the sacroiliac joint 800. The tip portion 450 may distract the sacroiliac joint 800 a specified distance sufficient to allow introduction of one or more prongs 520 of the forked dilator tube 500.

The cannulation 470 may be an internal diameter extending along the entire length of the straight chisel assembly 400 and generally along a longitudinal axis of the body 440. The cannulation 470 may be sized to slidably receive a k-wire 850.

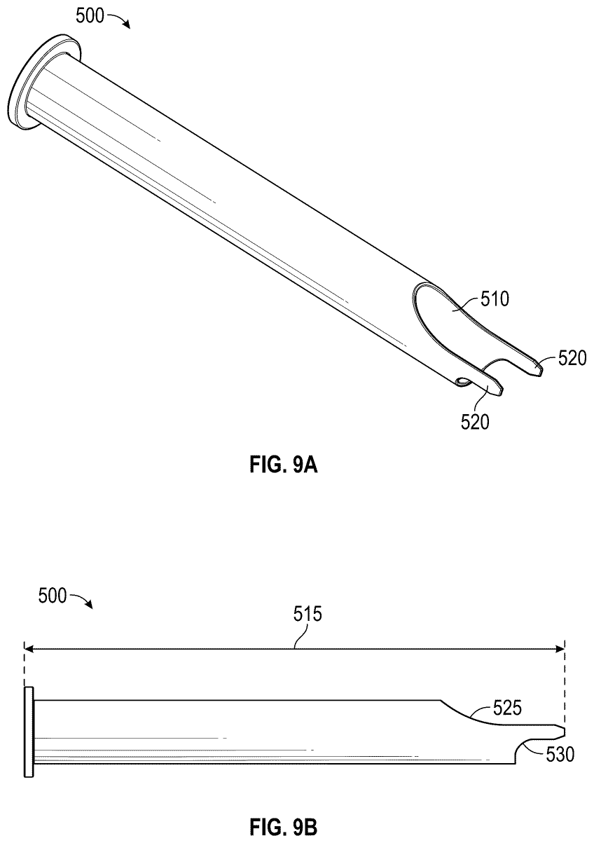

FIG. 9A is a perspective view of the forked dilator tube 500. FIG. 9B is a front view of the forked dilator tube 500. The forked dilator tube 500 may be configured the engage the sacroiliac joint 800 and provide a working channel for instruments and implants to access a desired implantation site of the sacroiliac joint 800. The forked dilator tube may include an inside diameter 510, a length 515, one or more prongs 520, a first clearance profile 525 and a second clearance profile 530.

The inside diameter 510 greater than or equal to the shaft diameter 455 of the straight chisel assembly 400. Additionally, the inside diameter 510 may be configured to receive the body 440 of the straight chisel assembly 400. The length 515 may be less than or equal to the shaft length 460 of the straight chisel assembly 400 so that the leading angle 457 of the tip portion 450 may extend beyond the one of more prongs 520 and engage the sacroiliac joint 800.

The one or more prongs 520 may be configured to penetrate the sacroiliac joint 800 in order to maintain a fixed spacing between the sacrum 810 and the ilium 820. The first clearance profile 525 may be configured so that the one or more prongs 520 of the forked dilator tube 500 may be introduced into the sacroiliac joint 800 without the ilium 820 interfering with the placement of the forked dilator tube 500. The second clearance profile 530 may be configured so that the one or more prongs 520 of the forked dilator tube 500 may be introduced into the sacroiliac joint 800 without the sacrum 810 interfering with the placement of the forked dilator tube 500.

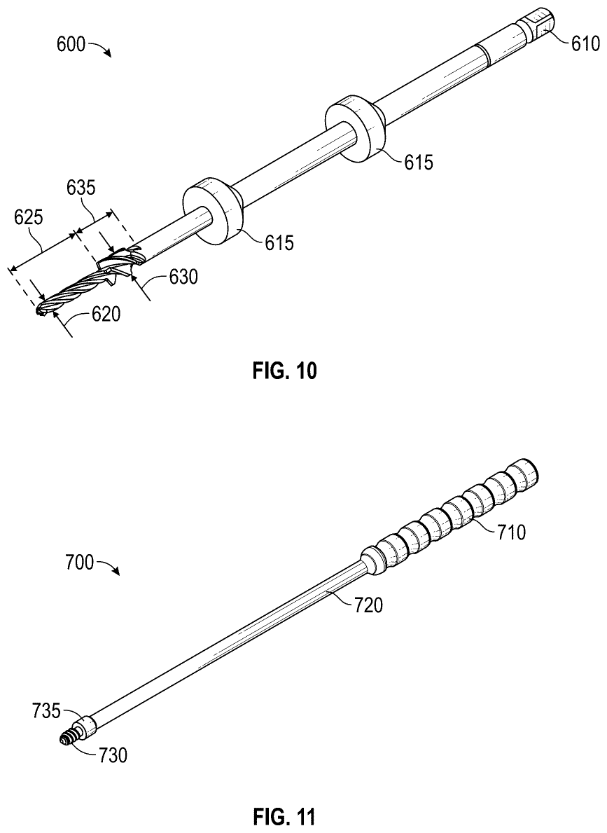

FIG. 10 is a perspective view of a step drill 600 according to an embodiment of the present disclosure. The step drill 600 may be configured to cut a first pilot hole and a second pilot hole whereby the first pilot hole is concentric with the second pilot hole and the first pilot hole has a smaller diameter than the second pilot hole. The step drill 600 may include a connection portion 610, one or more locating rings 615, a first cutting diameter 620, a first cutting length 625, a second cutting diameter 630, and a second cutting length.

The connection portion 610 may have with a male connection mechanism configured to releasably connect to a handle with a complimentary connection mechanism. The connection portion 610 may be configured with a quick connect style coupling mechanism. The connection portion 610 may be configured as one of: AO connector, Hudson connector, trilobe connector, square connector, hex connector, Stryker connector, Stryker-Hall connector, or other quick connect mechanism known in the art.

The one or more locating rings 615 may be received within the forked dilator tube 500 so that a longitudinal axis of the step drill 600 is generally colinear with a longitudinal axis of the forked dilator tube 500.

The first cutting diameter 620 may be configured to create a pilot hole in a portion of bone or a joint for the fastener 100. The first cutting diameter 620 may be less than or equal to a thread minor diameter of the fastener 100. The first cutting length 625 may be greater than or equal to a length of the threaded portion 130 of the fastener 100. The second cutting diameter 630 may be configured to create a pilot hole in a portion of bone or a joint for the Implant 200. The second cutting diameter 630 may be generally equal to a diameter of the body 205 of the Implant 200. The second cutting diameter 630 may leave material for the first wing 220, the second wing 222, the third wing 225, and/or the fourth wing 227 to dig into a portion of the sacrum 810 and/or the ilium 820.

The second cutting length 635 may be greater than or equal to the body height 255 of the Implant 200.

FIG. 11 is a perspective view of an Implant inserter 700 according to an embodiment of the present disclosure. The Implant inserter 700 may be configured to detachably connect to the Implant 200 so that the Implant 200 may be positioned on the sacroiliac joint 800 and embedded into the sacrum 810 and the ilium 820. The Implant inserter 700 may include a handle 710, a shaft 720, a connection portion 730, and a depth stop 735. The handle 710 and the shaft 720 may be constructed of two separate components and securably connected to each other. Alternatively, the handle 710 and the shaft 720 may be constructed as a single monolithic component.

The connection portion 730 may be configured to releasably connect the Implant inserter 700 to the Implant 200. The connection portion 730 may include a thread having a pitch and diameter that corresponds to the internal thread 215 of the Implant 200. The depth stop 735 may limit the depth of engagement between the connection portion 730 and the internal thread 215. Additionally, the depth stop 735 may provide a surface against which the body 205 of the Implant 200 may be tightened to ensure a secure connection between the Implant inserter 700 and the Implant 200.

The Implant inserter 700 may further be configured so that the handle may be impacted with a mallet or hammer to drive the Implant 200 into the sacrum 810 and/or the ilium 820.

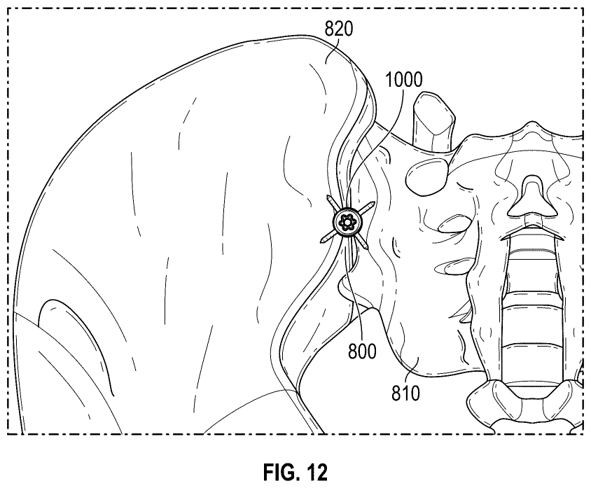

FIG. 12 is a front view of the sacroiliac joint stabilization system 1000 implanted in a sacroiliac joint 800. While a single sacroiliac joint stabilization system 1000 is shown at a single location along the sacroiliac joint 800, it should be noted that other configurations may be possible. For example, the sacroiliac joint stabilization system 1000 may be placed at a different location along the sacroiliac joint 800 shown. Additionally, or alternatively, additional sacroiliac joint stabilization systems 1000 may be placed at other locations along the sacroiliac joint 800 shown. Additionally, or alternatively, one or more sacroiliac joint stabilization systems 1000 may be placed along the opposite sacroiliac joint (not shown).

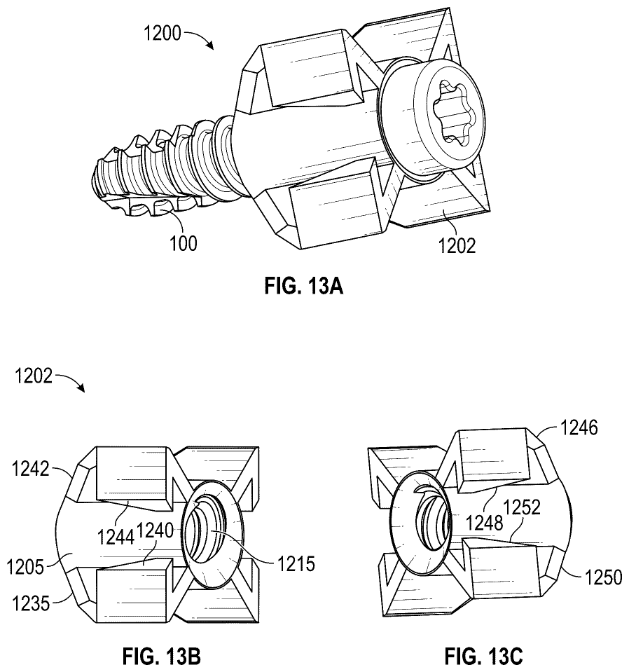

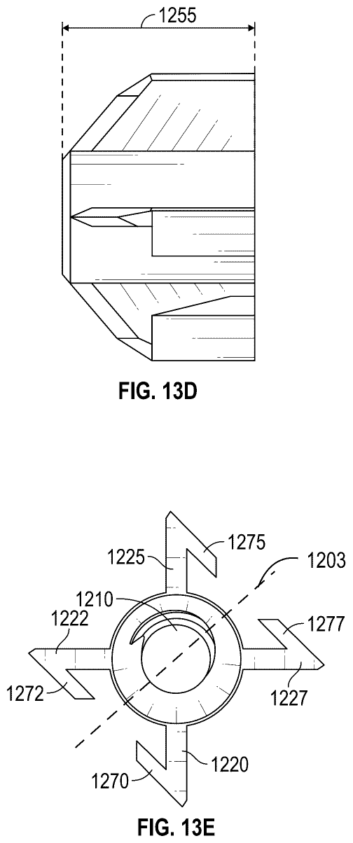

FIG. 13A is a perspective view of a sacroiliac joint stabilization system 1200 according to an embodiment of the present disclosure. The Implant 200 may be configured to span a sacroiliac joint 800 and engage a portion of a sacrum 810 and a portion of an ilium 820. The Implant 1202 may further be configured to minimize movement of an ilium 820 with respect to an adjacent sacrum 810. The Implant 1202 may be configured to urge the sacrum towards the ilium and the ilium towards the sacrum to compress the sacroiliac joint 800 when tapped into the sacroiliac joint 800.

Additionally, the Implant 1202 may be configured to receive a fastener 100 through an aperture 1210 in a body 1205. The Implant 1202 may further be configured to receive the fastener 100 through rotation of the fastener 100 but without rotation of the Implant 1202.

FIG. 13B is a perspective view of an Implant 1202 of the sacroiliac joint stabilization system 1200. FIG. 13C is an opposite perspective view of the Implant 1202, FIG. 13D is a side view of the Implant 1202, and FIG. 13E is a front view of the Implant 1202. The Implant 1202 may include a body 1205, an aperture 1210, an internal thread 1215, and a body height 1255.

The internal thread 1215 may include a thread pitch, thread form, and/or thread diameter compatible with the threaded portion 130 and thread diameter 140 of the fastener 100. The internal thread 1215 may be configured to allow the threaded portion 130 of the fastener 100 to threadably pass though the aperture 1210. The body height 1255 may be less than or equal to the shaft length 128 of the fastener 100 so that, after the threaded portion 130 of the fastener 100 passes through the aperture 1210, the shaft portion 125 of the fastener 100 may freely rotate within the Implant 1202 without the threaded portion 130 engaging the internal thread 1215. The body height 1255 may be between 10 mm and 15 mm. More specifically, the body height 1255 may be between 12 mm and 13 mm. Even more specifically, the body height 1255 may be approximately 12.5 mm.

The Implant 1202 may also include a first wing 1220 having a first sharp edge 1235 and a first sharp tapered edge 1240, a second wing 1222 having a second sharp edge 1242 and a second sharp tapered edge 1244, a third wing 1225 having a third sharp edge 1246 and a third sharp tapered edge 1248, and a fourth wing 1227 having a fourth sharp edge 1250 and a fourth sharp tapered edge 1252. The first sharp edge 1235, the second sharp edge 1242, the third sharp edge 1246, and the fourth sharp edge 1250 may be configured to penetrate a bone.

Each of the first wing 1220, the second wing 1222, the third wing 1225, and the fourth wing 1227 may extend radially from the body 1205. The first wing 1220, the second wing 1222, the third wing 1225, and the fourth wing 1227 may be generally equally spaced around a perimeter of the body whereby an angle between any two adjacent wings is approximately 90°.

Alternatively, a first angle between the first wing 1220 and the second wing 1222 and a second angle between the third wing 1225 and the fourth wing 1227 may each be less than 90° and a third angle between the second wing 1222 and the third wing 1225 and a fourth angle between the fourth wing 1227 and the first wing 1220 may each be greater than 90° whereby the sum of the first angle, the second angle, the third angle, and the fourth angle may equal 360°.

Additionally, or alternatively, a first angle between the first wing 1220 and the second wing 1222 may be different than a second angle between the third wing 1225 and the fourth wing 1227.

Additionally, or alternatively, one or more of the first wing 1220, the second wing 1222, the third wing 1225, and the fourth wing 1227 may be colinear with a secant of the body 1205 and not colinear with a diameter of the body 1205.

Additionally, or alternatively, the body 1205 may be configured as a polygon or other non-circular shape whereby each of the first wing 1220, the second wing 1222, the third wing 1225, and the fourth wing 1227 may extend from a face or a vertex of the polygon. In an embodiment, a first wing 1220 and a second wing 1222 may be positioned on a first side of a center plane 1203 that bisects the center of a body 1205, along a longitudinal axis of the body 1205, and a third wing 1225 and a fourth wing 1227 may be positioned on a second side of the center plane 1203.

The first wing 1220 may include a first angled portion 1270 that may form an acute angle relative to the first wing 1220 so that the first angled portion 1270 may be angled toward the second wing 1222. The second wing 1222 may include a second angled portion 1272 that may be at an acute angle relative to the second wing 1222 so that the second angled portion 1272 may be angled toward the first wing 1220.

The third wing 1225 may include a third angled portion 1275 that may be at an acute angle relative to the third wing 1225 so that the third angled portion 1275 may be angled toward the fourth wing 1227. The fourth wing 1227 may include a fourth angled portion 1277 that may be at an acute angle relative to the fourth wing 1227 so that the fourth angled portion 1277 may be angled toward the third wing 1225.

The first angled portion 1270 and the second angled portion 1272 may be generally colinear. The third angled portion 1275 and the fourth angled portion 1277 may be generally colinear. The first angled portion 1270 and the second angled portion 1272 may be generally parallel to the third angled portion 1275 and the fourth angled portion 1277. When the Implant 1202 is implanted to span the sacroiliac joint 800, the first angled portion 1270, the second angled portion 1272, the third angled portion 1275, and the fourth angled portion 1277 may be generally parallel to the sacroiliac joint 800.

The Implant 1202 may be configured to be embedded into the sacrum 810 and ilium 820 by orienting the Implant 1202 such that the first wing 1220 and the second wing 1222 face the sacrum 810 and the third wing 1225 and fourth wing 1227 face the ilium 820 and then pressing (or impacting) the Implant 1202 such that the first sharp edge 1235 and the second sharp edge 1242 penetrate the sacrum 810 and the third sharp edge 1246 and fourth sharp edge 1250 penetrate the ilium 820. The Implant 1202 may be further pressed (or impacted) until the Implant 1202 is fully embedded within the sacrum 810 and/or the ilium 820.

Alternatively, the Implant 1202 may not be fully embedded within the sacrum 810 and/or the ilium 820, the proximal end of the Implant 1202 may protrude above the surface of the sacrum 810 and/or the ilium 820. Additionally, or alternatively, the sacroiliac joint stabilization system 1200 may be configured so that, as the fastener 100 threadably engages a portion of bone, the head portion 110 exerts a linear force on the body 1205 that may result in embedding the Implant 1202 deeper into the sacrum 810 and/or the ilium 820.

The Implant 1202 may be positioned such that the acute angled formed between the first wing 1220 and the sacroiliac joint 800 may be generally equal to the acute angle formed between the second wing 1222 and the sacroiliac joint 800. Alternatively, the Implant 1202 may be positioned such that the acute angled formed between the first wing 1220 and the sacroiliac joint 800 is generally less than the acute angle formed between the second wing 1222 and the sacroiliac joint 800.

The first sharp tapered edge 1240, the second sharp tapered edge 1244, the third sharp tapered edge 1248, and the fourth sharp tapered edge 1252 may each be configured so that as the Implant 1202 is embedded into the sacrum 810 and ilium 820, the first angled portion 1270, the second angled portion 1272, the third angled portion 1275, and the fourth angled portion may urge the sacrum towards the ilium and the ilium towards the sacrum.

The first sharp tapered edge 1240, the second sharp tapered edge 1244, the third sharp tapered edge 1248, and the fourth sharp tapered edge 1252 may be angled so that as the implant 1202 is embedded into the sacrum 810 and the ilium 820, the first angled portion 1270, the second angled portion 1272, the third angled portion 1275, and the fourth angled portion are deflected away from the body 1205. The material properties of the implant 1202 may be selected so that the first angled portion 1270, the second angled portion 1272, the third angled portion 1275, and the fourth angled portion then apply a force to urge the sacrum towards the ilium and the ilium towards the sacrum.

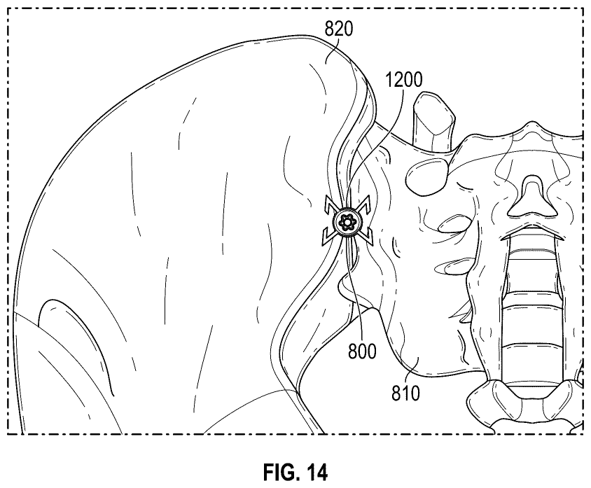

FIG. 14 is a front view of the sacroiliac joint stabilization system 1200 implanted in a sacroiliac joint 800. While a single sacroiliac joint stabilization system 1200 is shown at a single location along the sacroiliac joint 800, it should be noted that other configurations may be possible. For example, the sacroiliac joint stabilization system 1200 may be placed at a different location along the sacroiliac joint 800 shown. Additionally, or alternatively, additional sacroiliac joint stabilization systems 1200 may be placed at other locations along the sacroiliac joint 800 shown. Additionally, or alternatively, one or more sacroiliac joint stabilization systems 1200 may be placed along the opposite sacroiliac joint (not shown).

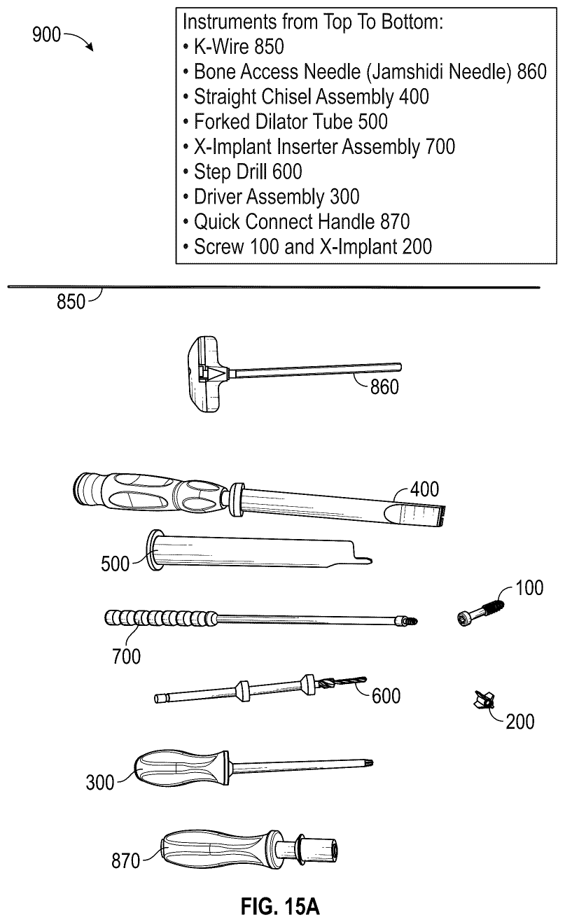

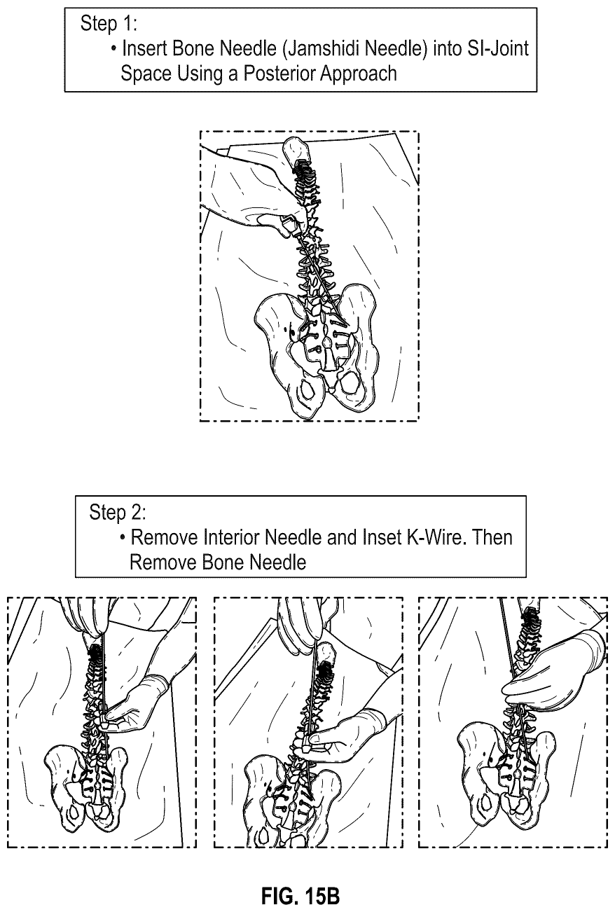

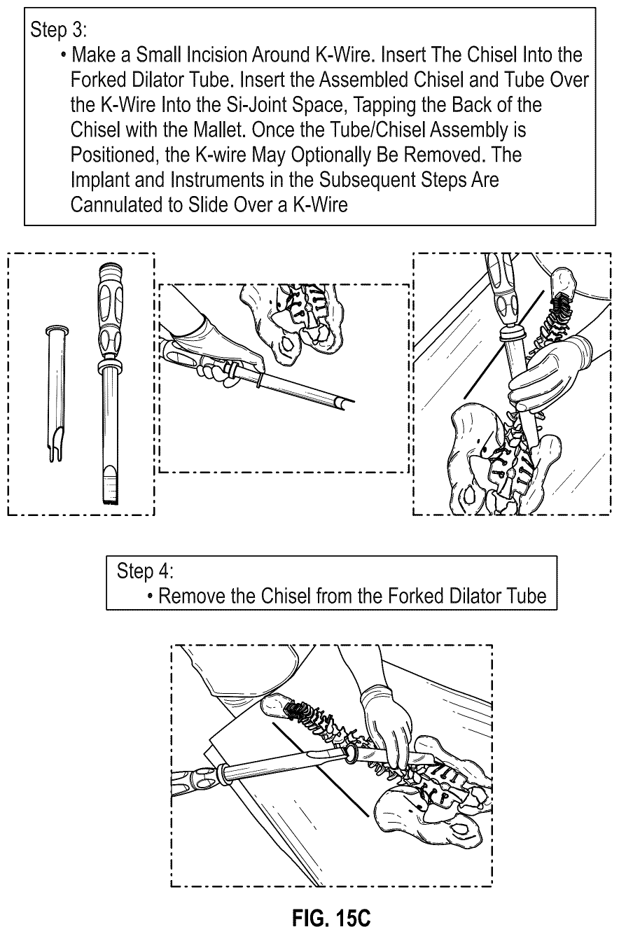

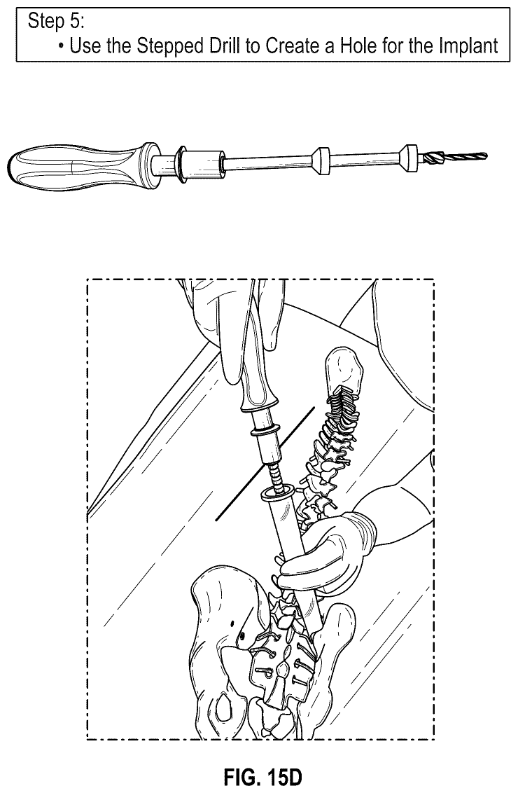

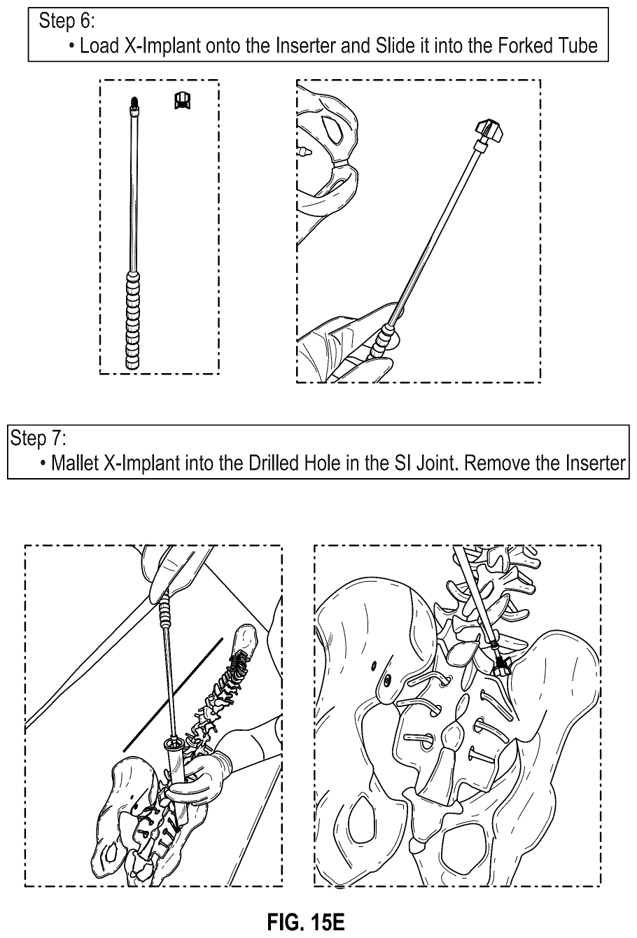

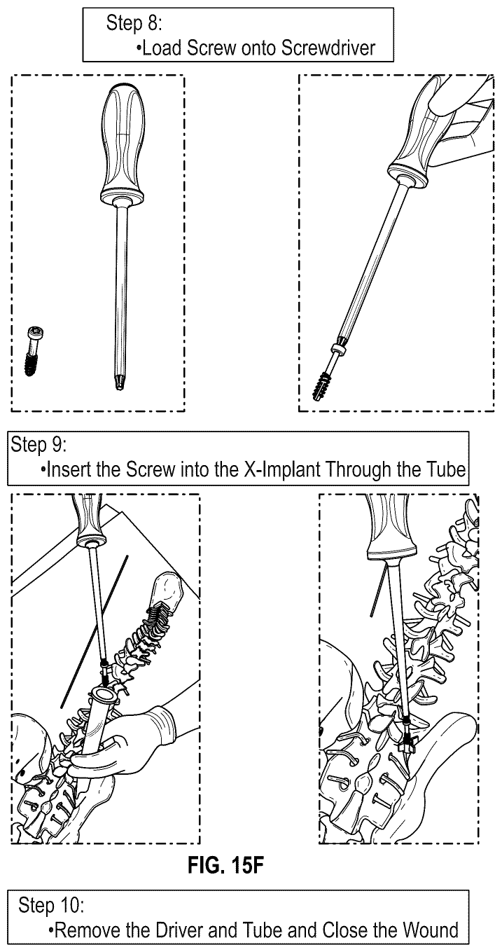

FIG. 15A through FIG. 15F are a method for implanting a sacroiliac joint stabilization system 1000 into a sacroiliac joint 800 according to an embodiment of the present disclosure. A method 900 for implanting a sacroiliac joint stabilization system 1000 may include a minimally invasive posterior surgical approach that may avoid nerves and large blood vessels. The method 900 for implanting a sacroiliac joint stabilization system, such as the sacroiliac joint stabilization system 1000, may include the following steps:

The method 900 may utilize the following instruments:
a K-wire 850
a bone access needle (Jamshidi needle) 860
a straight chisel assembly 400
a forked dilator tube 500
an Implant inserter 700
a step drill 600
a driver assembly 300
a quick connect handle 870
other standard surgical instruments known in the art

Step 1 may include inserting a Bone Needle (Jamshidi needle) 860 into a sacroiliac joint 800 space using a posterior approach.

Step 2 may include removing a bone needle 860 stylus and inserting a k-wire 850 through bone needle into the sacroiliac joint 800 space.

Step 3 may include making a small incision around the k-wire 850. Inserting the straight chisel assembly 400 into the forked dilator tube 500. Inserting the assembled straight chisel assembly 400 and forked dilator tube 500 over the K-wire 850 into the sacroiliac joint 800 space, tapping the back of the straight chisel assembly 400 with a mallet. Once the straight chisel assembly 400/forked dilator tube 500 assembly is positioned, the k-wire 850 may optionally be removed. The sacroiliac joint stabilization system 1000 and instruments in the subsequent steps are cannulated to slide over the k-wire 850.

Step 4 may include removing the straight chisel assembly 400 from the forked dilator tube 500.

Step 5 may include using the step drill 600 to create a hole for the sacroiliac joint stabilization system 1000.

Step 6 may include loading the Implant 200 onto the Implant inserter 700 and sliding the Implant 200 into the forked dilator tube 500.

Step 7 may include impacting the Implant inserter 700 with a mallet into the drilled hole in the sacroiliac joint 800 and then removing the Implant inserter 700.

Step 8 may include loading the fastener 100 onto the driver assembly 300.

Step 9 may include sliding the fastener 100 and driver assembly 300 into the forked dilator tube 500 and threadably advancing the fastener 100 through the Implant 200 and tightening the fastener 100 into the drilled hole.

Step 10 may include removing the driver assembly 300 and the forked dilator tube 500 and closing the wound with sutures.

Those of skill in the art will recognize that this is only one of many potential methods that may be used to implant a sacroiliac joint stabilization system, such as the sacroiliac joint stabilization system 1000. In alternative embodiments, different methods may be used to implant the sacroiliac joint stabilization system 1000 or other systems described above. Further, the method set forth in FIG. 15A though FIG. 15F may be used to implant other sacroiliac joint stabilization system systems besides those specifically disclosed herein.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

1. A sacroiliac joint stabilization system configured to stabilize a sacroiliac joint, the sacroiliac joint stabilization system comprising:
a fastener comprising a threaded portion; and
an implant comprising:
a body comprising a body height and an aperture extending along the body height;
a first wing;
a second wing;
a third wing; and
a fourth wing;
wherein:
the first wing, the second wing, the third wing, and the fourth wing are arranged relative to each other to define an X shape;
the threaded portion is configured to advance through the aperture and engage a sacrum and an ilium;
the first wing and the second wing are configured to be embedded in the sacrum; and
the third wing and the fourth wing are configured to be embedded in the ilium.

2. The sacroiliac joint stabilization system of claim 1, wherein the first wing, the second wing, the third wing and the fourth wing extend radially from the body.

3. The sacroiliac joint stabilization system of claim 2, wherein:
the first wing comprises a first angled portion forming a first acute angle relative to the first wing so that the first angled portion is angled toward the second wing;
the second wing comprises a second angled portion forming a second acute angle relative to the second wing so that the second angled portion is angled toward the first wing;
the third wing comprises a third angled portion forming a third acute angle relative to the third wing so that the third angled portion is angled toward the fourth wing; and
the fourth wing comprises a fourth angled portion forming a fourth acute angle relative to the fourth wing so that the fourth angled portion is angled toward the third wing.

4. The sacroiliac joint stabilization system of claim 3, wherein:
the first angled portion comprises a first sharp tapered edge;
the second angled portion comprises a second sharp tapered edge;
the third angled portion comprises a third sharp tapered edge;
the fourth angled portion comprises a fourth sharp tapered edge; and
the first sharp tapered edge, the second sharp tapered edge, the third sharp tapered edge, and the fourth sharp tapered edge are configured so that, with the implant embedded in the sacrum and the ilium, the first angled portion, the second angled portion, the third angled portion, and the fourth angled portion urge the sacrum towards the ilium and the ilium towards the sacrum.

5. The sacroiliac joint stabilization system of claim 1, wherein:
the first wing comprises a first sharp edge;
the second wing comprises a second sharp edge;
the third wing comprises a third sharp edge;
the fourth wing comprises a fourth sharp edge;
the first sharp edge and the second sharp edge are configured to penetrate the sacrum; and
the third sharp edge and the fourth sharp edge are configured to penetrate the ilium.

6. The sacroiliac joint stabilization system of claim 1, wherein the fastener further comprises a head portion comprising a drive feature, and a shaft portion between the threaded portion and the head portion, wherein the aperture is configured to receive the shaft portion, and, with the shaft portion received in the aperture, the fastener is rotatable relative to the implant.

7. The sacroiliac joint stabilization system of claim 1, wherein:
the first wing comprises a first height that is generally equal to the body height;
the second wing comprises a second height that is generally equal to the body height;
the third wing comprises a third height that is generally equal to the body height; and
the fourth wing comprises a fourth height that is generally equal to the body height.

8. A sacroiliac joint stabilization system configured to stabilize a sacroiliac joint between a sacrum and an ilium, the sacroiliac joint stabilization system comprising:
a fastener comprising:
a threaded portion;
a head portion comprising a drive feature; and
a shaft portion between the threaded portion and the head portion; and
an implant comprising:
a body comprising a body height;
an aperture extending along the body height and comprising an internal thread;
a first wing comprising a first height that is generally equal to the body height;
a second wing comprising a second height that is generally equal to the body height;
a third wing comprising a third height that is generally equal to the body height; and
a fourth wing comprising a fourth height that is generally equal to the body height;
wherein:
the first wing, the second wing, the third wing and the fourth wing are arranged relative to each other to define an X shape;
the threaded portion is configured to engage the internal thread and threadably advance through the aperture;
the aperture is configured to receive the shaft portion; and
with the shaft portion received in the aperture, the fastener is rotatable relative to the implant.

9. The sacroiliac joint stabilization system of claim 8, wherein:
the threaded portion is configured to advance through the aperture and engage the sacrum and the ilium;
the first wing and the second wing are configured to be embedded in the sacrum; and
the third wing and the fourth wing are configured to be embedded in the ilium.

10. The sacroiliac joint stabilization system of claim 8, wherein the first wing, the second wing, the third wing and the fourth wing extend radially from the body.

11. The sacroiliac joint stabilization system of claim 10, wherein:
the first wing comprises a first angled portion forming a first acute angle relative to the first wing so that the first angled portion is angled toward the second wing;
the second wing comprises a second angled portion forming a second acute angle relative to the second wing so that the second angled portion is angled toward the first wing;
the third wing comprises a third angled portion forming a third acute angle relative to the third wing so that the third angled portion is angled toward the fourth wing; and
the fourth wing comprises a fourth angled portion forming a fourth acute angle relative to the fourth wing so that the fourth angled portion is angled toward the third wing.

12. The sacroiliac joint stabilization system of claim 11, wherein:
the first angled portion comprises a first sharp tapered edge;
the second angled portion comprises a second sharp tapered edge;
the third angled portion comprises a third sharp tapered edge;
the fourth angled portion comprises a fourth sharp tapered edge; and
the first sharp tapered edge, the second sharp tapered edge, the third sharp tapered edge, and the fourth sharp tapered edge are configured so that, with the implant embedded in the sacrum and the ilium, the first angled portion, the second angled portion, the third angled portion, and the fourth angled portion urge the sacrum towards the ilium and the ilium towards the sacrum.

13. The sacroiliac joint stabilization system of claim 8, wherein:
the first wing comprises a first sharp edge;
the second wing comprises a second sharp edge;
the third wing comprises a third sharp edge;
the fourth wing comprises a fourth sharp edge;
the first sharp edge and the second sharp edge are configured to penetrate the sacrum; and
the third sharp edge and the fourth sharp edge are configured to penetrate the ilium.

14. The sacroiliac joint stabilization system of claim 8, wherein the threaded portion is configured to advance through the aperture and engage the sacrum and the ilium.

15. A sacroiliac joint stabilization system configured to stabilize a sacroiliac joint between a sacrum and an ilium, the sacroiliac joint stabilization system comprising:
an implant comprising:
a first wing comprising a first sharp edge;
a second wing comprising a second sharp edge;
a third wing comprising a third sharp edge; and
a fourth wing comprising a fourth sharp edge;
wherein:
the first wing, the second wing, the third wing and the fourth wing are arranged relative to each other to define an X shape;
the first sharp edge and the second sharp edge are configured to penetrate the sacrum; and
the third sharp edge and the fourth sharp edge are configured to penetrate the ilium.

16. The sacroiliac joint stabilization system of claim 15, wherein the first wing and the second wing are configured to be embedded in the sacrum, and the third wing and the fourth wing are configured to be embedded in the ilium.

17. The sacroiliac joint stabilization system of claim 16, wherein, with the first wing and the second wing embedded in the sacrum, and the third wing and the fourth wing embedded in the ilium, the implant is configured to urge the sacrum towards the ilium, and the ilium towards the sacrum.

18. The sacroiliac joint stabilization system of claim 15, wherein the implant further comprises a body comprising an aperture extending along a body height of the body,
wherein:
the first wing, the second wing, the third wing and the fourth wing extend radially from the body;
the first wing comprises a first height that is generally equal to the body height;
the second wing comprises a second height that is generally equal to the body height;
the third wing comprises a third height that is generally equal to the body height; and
the fourth wing comprises a fourth height that is generally equal to the body height.

19. The sacroiliac joint stabilization system of claim 18, wherein:
the first wing comprises a first angled portion forming a first acute angle relative to the first wing so that the first angled portion is angled toward the second wing;
the second wing comprises a second angled portion forming a second acute angle relative to the second wing so that the second angled portion is angled toward the first wing;
the third wing comprises a third angled portion forming a third acute angle relative to the third wing so that the third angled portion is angled toward the fourth wing; and
the fourth wing comprises a fourth angled portion forming a fourth acute angle relative to the fourth wing so that the fourth angled portion is angled toward the third wing.

20. The sacroiliac joint stabilization system of claim 15, further comprising a fastener comprising a threaded portion, a head portion comprising a drive feature, and a shaft portion between the threaded portion and the head portion, wherein:
the implant further comprises a body comprising an aperture extending along a body height of the body;
the aperture is configured to receive the shaft portion; and
with the shaft portion received in the aperture, the fastener is rotatable relative to the implant.
